(19) United States
(12) Patent Application Publication
(10) Pub. No.: US 2020/0170369 A1
(43) Pub. Date: Jun. 4, 2020

(54) PROCESS FOR TREATING KERATIN FIBERS USING A MONOSACCHARIDE WITH AMINE GROUP, A POLYSACCHARIDE WITH AMINE GROUP AND A STEP OF HEAT TREATMENT

(71) Applicant: L'OREAL, Paris (FR)

(72) Inventors: Andrew GREAVES, AULNAY-SOUS-BOIS (FR); Nawel BAGHDADLI, AULNAY-SOUS-BOIS (FR); Bérangère BARIL, AULNAY-SOUS-BOIS (FR); Lucien BILDSTEIN, AULNAY-SOUS-BOIS (FR)

(21) Appl. No.: 16/641,144

(22) PCT Filed: Aug. 29, 2018
(86) PCT No.: PCT/EP2018/073183

§ 371 Date: Feb. 21, 2020

(30) Foreign Application Priority Data
Aug. 31, 2017 (FR) 1758048

(51) Int. Cl. A45D 7/06; A61K 8/60; A61K 8/73; A61K 8/36; A61Q 5/00; A61Q 5/06

(52) U.S. Cl. CPC A45D 7/06; A61K 8/60; A61K 8/736; A61K 2800/884; A61Q 5/002; A61Q 5/06; A61K 2800/30; A61K 8/36

(57) ABSTRACT

The invention relates to i) a process for treating keratin fibres, in particular human keratin fibres such as the hair, employing a) at least one monosaccharide with amine group(s), b) at least one polysaccharide with amine group(s) then c) a heat treatment; ii) a composition having an acid pH comprising the ingredients a), b) and c) at least one organic or mineral acid a), b) and c) then heat to treat keratin fibres, in particular human keratin fibres such as the hair, and iv) a multi-compartment kit or device comprising a) and b). The invention makes it possible to improve the cosmetic effect of the keratin fibres intrinsically, in order to prevent or reduce the breakage of natural keratin fibres and/or to repair damaged keratin fibres, while retaining or even improving the manageability, anti-frizz properties and/or persistence of the styling of said fibres, in particular human fibres such as the hair, when a heating step is carried out.

The invention relates to i) a process for treating keratin fibres, in particular human keratin fibres such as the hair, employing a) at least one monosaccharide with amine group(s), b) at least one polysaccharide with amine group(s) then a heat treatment or heat (iii); ii) a composition having an acid pH (C) comprising the ingredients a) and b) in salified or non-salified form; iii) the use of the ingredients a) and b) then of heat (iii) to treat the keratin fibres, in particular human keratin fibres such as the hair, and iv) a multi-compartment kit or device comprising a) and b).

Hair is generally damaged and weakened by the action of external atmospheric agents such as light, sun and bad weather, and also by mechanical or chemical treatments, such as brushing, combing, dyeing, bleaching, permanent-waving, relaxing and repeated washing. Hair is thus damaged by these various factors and may in the long run become dry, coarse, brittle, dull, split and/or limp.

Thus, to overcome these drawbacks, it is common practice to resort to hair treatments which make use of compositions intended for conditioning the hair appropriately by giving it satisfactory cosmetic properties, especially smoothness, sheen, a soft feel (a natural feel; the hair is no longer coarse), suppleness, a lightweight feel, good disentangling properties leading to easy combing, and good manageability of the hair which is thus easy to shape.

These hair care compositions can, for example, be conditioning shampoos, hair conditioners, masks or serums. However, the conditioning effect obtained fades out in the course of successive shampoo washes and does not show satisfactory persistence on shampooing.

It is known practice to employ care compositions comprising reducing sugars such as monosaccharides, used as conditioning agents, especially to repair keratin fibres which have been damaged by harsh treatments.

Indeed, patent application US 2002/0193264 describes a process for conditioning keratin fibres, in which at least one sugar chosen from monosaccharides is applied to said fibres, and a step of heating the keratin fibres is carried out. Similarly, patent application US 2002/0172653 discloses a process for conditioning keratin fibres comprising a step of applying to said fibres a sugar chosen from specific C₅-C₇ monosaccharides and a step of heating the keratin fibres. However, the use of reducing sugars followed by a heat treatment may lead to an undesired modification of the colour of the keratin fibres. Thus, the keratin fibres are not always sufficiently protected, repaired or cosmetically transformed in a long-lasting manner. Another document describes the use of oxidized polysaccharide with an amine-containing monosaccharide (WO 2016/142551). Nevertheless, the results obtained are not always satisfactory in terms of repairing or preventing breakage of the keratin fibres, in particular split keratin fibres, when a treatment with heat is carried out.

There is therefore a real need to develop a composition and a process for treating keratin fibres, in particular human keratin fibres such as the hair, which is able to protect and/or repair said fibres, if possible in a lasting manner, with respect to external agents and/or heat.

Moreover, it is advantageous to find a means for treating damaged keratin fibres by repairing them, that is to say by improving the cosmetic effect of damaged keratin fibres intrinsically, while retaining or even improving the manageability, the anti-frizz properties and/or the persistence of the styling of said fibres. It is also advantageous to prevent the breakage of natural keratin fibres, while retaining or even improving the manageability, the anti-frizz properties and/or the persistence of the styling of said fibres.

This (these) technical problem(s) is (are) solved by the process for treating keratin fibres, especially human keratin fibres, in particular the hair, comprising:
(i) a step consisting in applying, to said fibres, a) one or more monosaccharides with amine group(s);
(ii) a step consisting in applying, to said fibres, b) one or more polysaccharides with amine group(s), preferably having an average molecular weight of less than or equal to 400 kDa;
(ii′) optionally a drying step;
(iii) then a step of heat treatment at a temperature above or equal to 80° C., in particular
   at a temperature between 100° C. and 250° C., preferably with a hair iron; it being understood that the steps (i) and (ii) may be carried out simultaneously, or sequentially, preferably the steps (i) and (ii) are carried out simultaneously and that the drying step (ii′), when it is present, precedes the heat treatment step and follows the steps (i) and (ii).

Another subject of the invention is an aqueous composition having an acid pH (pH<7), composition also referred to as (C), comprising:

either
a) one or more salified monosaccharides with amine group(s); and b) one or more salified polysaccharides with amine group(s), preferably having an average molecular weight of less than or equal to 400 kDa, and optionally c) one more mineral or organic acids, preferably different from the following acids: aspartic acid, glutamic acid, gluconic acid, pyrrolidonecarboxylic acid, 100 OE and 500 OE ethoxylated stearic acid, linoleic acid, succinic acid;
   or
a) one or more non-salified monosaccharides with amine group(s); and
b) one or more salified polysaccharides with amine group(s), preferably having an average molecular weight of less than or equal to 400 kDa, and
and optionally c) one or more mineral or organic acids preferably different from the following acids: aspartic acid, glutamic acid, gluconic acid, pyrrolidonecarboxylic acid, 100 OE and 500 OE ethoxylated stearic acid, linoleic acid, succinic acid and alginic acid;
   or a) one or more salified monosaccharides with amine group(s); and
b) one or more non-salified polysaccharides with amine group(s), preferably having an average molecular weight of less than or equal to 400 kDa,
and optionally c) one or more mineral or organic acids preferably different from the following acids: aspartic acid, glutamic acid, gluconic acid, pyrrolidonecarboxylic acid, 100 OE and 500 OE ethoxylated stearic acid, linoleic acid, succinic acid and alginic acid;
   or
a) one or more non-salified monosaccharide(s) with amine group(s);
b) one or more non-salified polysaccharide(s) with amine group(s), preferably having an average molecular weight of less than or equal to 400 kDa, and
c) one or more mineral or organic acids different from the following acids: aspartic acid, glutamic acid, gluconic acid, pyrrolidonecarboxylic acid, 100 OE and 500 OE ethoxylated stearic acid, linoleic acid, succinic acid and alginic acid.

Another subject of the invention is the use of the ingredients a), b) and optionally c) as defined previously to treat keratin fibres, in particular to improve the cosmetic effect of the keratin fibres intrinsically, to prevent or reduce the breakage of natural keratin fibres and/or to repair damaged keratin fibres, while retaining or even improving the manageability, anti-frizz properties and/or the persistence of the styling of said fibres, in particular human fibres such as the hair, when a fibre heating step (iii) is carried out at at least 80° C., in particular at a temperature between 100° C. and 250° C., preferably with a hair iron.

Another subject is the use of the composition having an acid pH (C) as defined previously to improve the cosmetic effect of the keratin fibres intrinsically, to prevent or reduce the breakage of natural keratin fibres and/or to repair damaged keratin fibres, while retaining or even improving the manageability, anti-frizz properties and/or the persistence of the styling of said fibres, in particular human fibres such as the hair, when a fibre heating step is carried out, especially at a temperature above 80° C., in particular between 80° C. and 250° C., preferably with a hair iron.

Another subject of the invention is a cosmetic treatment process using the composition having an acid pH (C) as described previously to improve the cosmetic effect of the keratin fibres intrinsically, to prevent or reduce the breakage of natural keratin fibres and/or to repair damaged keratin fibres, while retaining or even improving the manageability, anti-frizz properties and/or the persistence of the styling of said fibres, in particular human fibres such as the hair, when a fibre heating step is carried out, especially at a temperature above 80° C., in particular between 80° C. and 250° C., preferably with a hair iron.

Another subject of the invention is a process for treating keratin fibres, especially human keratin fibres, in particular the hair, comprising:
(i) a step consisting in applying, to said fibres, a cosmetic composition (ca) containing
   a) one or more monosaccharides with amine group(s);
(ii) a step consisting in applying, to said fibres, a cosmetic composition (cb) containing
   b) one or more polysaccharides with amine group(s), preferably having an average molecular weight of less than or equal to 400 kDa;
(ii′) optionally a drying step;
(iii) then a step of heat treatment at a temperature above or equal to 80° C., in particular at a temperature between 100° C. and 250° C., preferably with a hair iron; it being understood that the steps (i) and (ii) may be carried out simultaneously, or sequentially, preferably the steps (i) and (ii) are carried out simultaneously and that the drying step (ii′), when it is present, precedes the heat treatment step and follows the steps (i) and (ii).

In particular, hair treated with the method according to the invention remains well-behaved since the presence of frizziness is not observed. Thus, the hairs are aligned, smooth and disentangle easily, which makes them easier to comb. The treated hair also has more body (it is not limp) and is thus easier to style. The treated hair is easy to shape, this shaping of the hair persisting even after several shampoo washes. Moreover, the treated hair is also shiny and soft to the touch. It is stronger and less brittle. The process according to the invention has the advantage of giving good persistence of these good hair-conditioning cosmetic properties after at least one shampoo wash, in particular after 5 successive shampoo washes. Thus, the treated hair is conditioned in a long-lasting manner. After treatment, the hair is not laden, and has a natural feel. The process, the composition (C) and the use of the invention make it possible to repair the split ends of keratin fibres, especially human keratin fibres such as the hair. Moreover, the process, the composition (C) and the use of the invention make it possible to protect the synthetic (oxidation and direct) dyeing of the keratin fibres, to reduce the fading of the colour of said fibres linked in particular to successive shampoo washes, and/or to repeated hair iron treatments, especially straightening iron treatments, without damaging said fibres.

In the following text, unless indicated otherwise:
the term “monosaccharides” is understood to mean a monosaccharide sugar comprising at least 5 carbon atoms of formula Cₓ(H₂O)ₓ with x an integer greater than or equal to 5, preferably x is greater than or equal to 6, in particular x is between 5 and 7 inclusive, preferably x=6, they may be of D or L configuration, and of alpha or beta anomer, and also the salts thereof and the solvates thereof such as hydrates;
the term “polysaccharides” is understood to mean a polysaccharide sugar which is a polymer constituted of several saccharides bonded to one another by O-glucosidic bonds, said polymers being constituted of monosaccharide units as defined previously, said monosaccharide units comprising at least 5 carbon atoms, preferably 6, particularly the monosaccharide units are connected to one another by 1,4 or 1,6 bond as α (alpha) or β (beta) anomer, it being possible for each saccharide unit to be of L or D configuration, and also the salts thereof and the solvates thereof such as the hydrates of said monosaccharides; more particularly they are polymers formed of a certain number of saccharides (or monosaccharides) having the general formula: —[Cₓ(H₂O)_{y})]ₙ— where x is an integer greater than or equal to 5, preferably x is greater than or equal to 6, in particular x is between 5 and 7 inclusive and preferably x=6, and y is an integer which represents x−1, and n is an integer greater than or equal to 2, particularly of between 3 and 3000 inclusive, more particularly between 5 and 2500 and preferentially between 10 and 2300;
the term “with amine group(s)” is understood to mean that the monosaccharide(s) a) and the polysaccharide(s) b) is (are) substituted with one or more NR₁R₂ amino group(s) i.e. at least one of the hydroxyl groups of at least one saccharide unit is replaced with an NR₁R₂ group with R₁ and R₂, which are identical or different, representing i) a hydrogen atom, ii) a (C₁-C₆)alkyl group that is optionally substituted, preferably with one or more hydroxyl or NH₂ groups, iii) an aryl group such as phenyl, iv) an aryl(C₁-C₄)alkyl group such as benzyl, v) a (hetero)cyclo(C₅-C₇)alkyl group such as cyclohexyl, morpholinyl, piperazinyl, piperidinyl, vi) a (hetero)cyclo(C₅-C₇)alkyl(C₁-C₄)alkyl group such as cyclohexylmethyl, vii) —C(Y)—(Y′)ₚ—R′1 with Y and Y′, which are identical or different, representing an oxygen atom, sulfur atom or N(R′2), preferably oxygen, p=0 or 1, preferably 0; and R′₁ and R′₂ representing i) to vi) of R₁ and R₂ defined previously, and in particular R′₁ denoting a (C₁-C₆)alkyl group such as methyl. Preferably R₁ and R₂ represent a hydrogen atom or a (C₁-C₄)alkylcarbonyl group such as acetyl;
the term “low molecular weight” is understood to mean that the polysaccharide with amine group has an average molecular weight of less than 400 000 Da;
the term “organic or mineral acid salt” is more particularly understood to mean organic or mineral acid salts in particular chosen from a salt derived from i) hydrochloric acid HCl, ii) hydrobromic acid HBr, iii) sulfuric acid H₂SO₄, iv) alkylsulfonic acids: Alk-S(O)₂OH such as methylsulfonic acid and ethylsulfonic acid; v) arylsulfonic acids: Ar—S(O)₂OH such as benzenesulfonic acid and toluenesulfonic acid; vi) alkoxysulfinic acids: Alk-O—S(O)OH such as methoxysulfinic acid and ethoxysulfinic acid; vii) aryloxysulfinic acids such as tolueneoxysulfinic acid and phenoxysulfinic acid; viii) phosphoric acid H₃PO₄; ix) triflic acid CF₃SO₃H and x) tetrafluoroboric acid HBF₄; xi) organic monocarboxylic acids (I) R—C(O)—OH with R representing a (hetero)aryl group such as phenyl, a (hetero)aryl(C₁-C₄)alkyl such as benzyl, or (C₁-C₃₀)alkyl or an unsaturated C₂-C₃₀ radical (i.e. comprising at least one ethylenic unsaturation, preferably one ethylenic unsaturation) said alkyl group or unsaturated C₂-C₃₀ radical being optionally interrupted and/or optionally substituted preferably with one or more hydroxyl groups and not substituted with one or more amino radicals, R preferably denoting a (C₁-C₆)alkyl group optionally interrupted and/or optionally substituted with 1, 2 or 3 hydroxyl groups, preferably R represents a (C₁-C₄)alkyl group such as methyl or ethyl; in particular the organic monocarboxylic acids (I) are chosen from acetic acid, glycolic acid and lactic acid, more particularly from acetic acid and lactic acid, and xii) the polycarboxylic acids of formula (II) below:

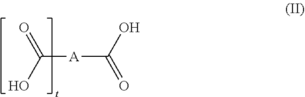

in which formula (II):
A represents a saturated or unsaturated, cyclic or noncyclic, aromatic or nonaromatic, polyvalent hydrocarbon-based group comprising from 1 to 30 carbon atoms optionally interrupted with one or more heteroatoms such as oxygen and/or optionally substituted in particular with one or more hydroxyl groups and t represents an integer between 1 and 5 inclusive; preferably A represents a divalent (C₁-C₆)alkylene group optionally substituted in particular with one or more hydroxyl groups and not substituted with at least one amino radical, and t is equal to 1, 2 or 3, the polycarboxylic acids of formula (II) are preferably chosen from tartaric acid, succinic acid, fumaric acid and citric acid and more particularly chosen from tartaric acid, succinic acid, fumaric acid, maleic acid and citric acid; and xiii) amino acids comprising more carboxylic acid radicals than amino groups such as gamma-carboxyglutamic acid, aspartic acid, glutamic acid, in particular gamma-carboxyglutamic acid; in particular the salts of monocarboxylic acids different from pyrrolidonecarboxylic acid, 100 OE and 500 OE ethoxylated stearic acid and linoleic acid, more particularly of monocarboxylic acid of formula;
an “alkyl radical” is a linear or branched C₁-C₁₀, preferably C₁-C₆ and in particular C₁-C₄ hydrocarbon-based radical, such as methyl or ethyl, unless otherwise indicated;
a (Cₓ-C_{y})alkyl radical is a linear or branched Cₓ-C_{y} hydrocarbon-based radical;
the expression “optionally interrupted” attributed to the alkyl radical or to the polyvalent group A defined previously is understood to mean that said radical may be interrupted with one or more groups or heteroatoms chosen from O, S, CO or combinations thereof such as —CO—O— or —O—CO—, preferably interrupted with one or more non-adjacent oxygen atoms;
the expression “optionally substituted” attributed to the alkyl radical or to the polyvalent group A defined previously is understood to mean that said radical may be substituted with one or more radicals chosen from the following radicals i) hydroxyl, ii) C₁-C₄ alkoxy, iii) acylamino, iv) amino optionally substituted with one or two identical or different C₁-C₄ alkyl radicals, said alkyl radicals possibly forming, with the nitrogen atom that bears them, a 5- to 7-membered heterocycle, optionally comprising another nitrogen or non-nitrogen heteroatom;
an “alkoxy radical” is an alkyl-oxy radical for which the alkyl radical is a linear or branched C₁-C₁₀, preferentially C₁-C₆ and more particularly C₁-C₄ hydrocarbon-based radical such as methoxy or ethoxy;
when the alkoxy group is optionally substituted, this implies that the alkyl group is optionally substituted as defined hereinabove;
the “aryl” or “heteroaryl” radicals or the aryl or heteroaryl part of a radical may be substituted with at least one substituent borne by a carbon atom, chosen from one of the following atoms or groups:
   halogen;
   optionally substituted C₁-06, preferably C₁-04, alkyl;
   hydroxyl;
   C₁-C₂ alkoxy;
   C₁-04 (poly)hydroxyalkoxy;
   amino;
   5- or 6-membered heterocycloalkyl;
   5- or 6-membered heteroaryl, optionally substituted with a (C₁-C₄)alkyl radical, preferentially methyl;
   amino substituted with one or two optionally substituted, identical or different C₁-C₆ alkyl radicals:
   acylamino (—NR—C(O)—R′) in which the R radical is a hydrogen atom or a C₁-C₄ alkyl radical optionally bearing at least one hydroxyl group and the R′ radical is a C₁-C₂ alkyl radical;
carbamoyl ((R)₂N—C(O)—) in which the R radicals, which may be identical or different, represent a hydrogen atom or a C₁-C₄ alkyl radical optionally bearing at least one hydroxyl group;
alkylsulfonylamino (R′—S(O)₂—N(R)—) in which the R radical represents a hydrogen atom or a C₁-C₄ alkyl radical optionally bearing at least one hydroxyl group and the radical R′ represents a C₁-C₄ alkyl radical, or a phenyl radical;
aminosulfonyl ((R)₂N—S(O)₂—) in which the R radicals, which may be identical or different, represent a hydrogen atom or a C₁-C₄ alkyl radical optionally bearing at least one hydroxyl group;
cyano;
nitro or nitroso; and
polyhaloalkyl, preferentially trifluoromethyl;
   the cyclic or heterocyclic part of a non-aromatic group may be substituted with at least one substituent chosen from the following groups:
hydroxyl;
C₁-C₄ alkoxy or C₂-04 (poly)hydroxyalkoxy;
C₁-C₄ alkyl;
alkylcarbonylamino (R—C(O)—N(R′)—), in which the R′ radical is a hydrogen atom or a C₁-C₄ alkyl radical optionally bearing at least one hydroxyl group and the R radical is a C₁-C₂ alkyl group or an amino optionally substituted with one or two identical or different C₁-C₄ alkyl groups themselves optionally bearing at least one hydroxyl group;
alkylcarbonyloxy (R—C(O)—O—), in which the R radical is a C₁-C₄ alkyl radical or an amino group optionally substituted with one or two identical or different C₁-C₄ alkyl groups themselves optionally bearing at least one hydroxyl group;
alkoxycarbonyl (R-G-C(O)—), in which the R radical is a C₁-C₄ alkoxy radical and G is an oxygen atom, or an amino group optionally substituted with a C₁-C₄ alkyl group itself optionally bearing at least one hydroxyl group;
a cyclic or heterocyclic radical, or a non-aromatic part of an aryl or heteroaryl radical, may also be substituted with one or more oxo groups;
an “aryl” radical represents a monocyclic or fused or non-fused polycyclic carbon-based group, comprising from 6 to 22 carbon atoms, at least one ring of which is aromatic; such as phenyl, biphenyl, naphthyl, indenyl, anthracenyl or tetrahydronaphthyl, preferentially phenyl;
a “heteroaryl radical” represents an optionally cationic, 5- to 22-membered, monocyclic or fused or non-fused polycyclic group, comprising from 1 to 6 heteroatoms chosen from nitrogen, oxygen, sulfur and selenium atoms, at least one ring of which is aromatic; preferentially, a heteroaryl radical is chosen from acridinyl, (benz)imidazolyl, (benzo)bistriazolyl, (benzo)pyrazolyl, (benzo)pyridazinyl, (benzo)quinolyl, (benzo)thiazolyl, (benzo)triazolyl, (benz)oxazolyl, pyridyl, tetrazolyl, dihydrothiazolyl, imidazopyridyl, indolyl, isoquinolyl, naphthoimidazolyl, naphthoxazolyl, naphthopyrazolyl, oxadiazolyl, oxazolyl, oxazolopyridyl, phenazinyl, phenoxazolyl, pyrilyl, pyrazoyltriazyl, pyridyl, pyridinoimidazolyl, pyrrolyl, quinolyl, tetrazolyl, thiadiazolyl, thiazolopyridyl, thiazoylimidazolyl, thiopyrylyl, triazolyl and xanthyl;
a “heterocyclic radical” is a 5- to 22-membered monocyclic or fused or non-fused polycyclic radical comprising one or two unsaturations but is non-aromatic, comprising from 1 to 6 heteroatoms chosen from nitrogen, oxygen, sulfur and selenium atoms;
a “heterocycloalkyl radical” is a heterocyclic radical comprising at least one saturated ring;
“limp” keratin fibres or “limp” hair is understood to mean that said fibres or hair are elastic, have no body, do not hold shape, the head of hair is flat without volume;
the expression “at least one” is equivalent to “one or more”; and
the expression “inclusive” for a range of concentrations means that the limits of the range are included in the defined interval.

a) Monosaccharides with Amine Group(s)

The first ingredient used in the present invention is a) one or more monosaccharides with amine group(s), and also the organic or mineral acid salts thereof, the α or β anomers thereof, the optical isomers thereof of L or D configuration, and the solvates thereof such as hydrates.

Preferably the monosaccharide(s) with amine group(s) of the invention a) is (are) salified with the aid of a mineral acid such as hydrochloric acid.

According to one particular form of the invention, the monosaccharide(s) with amine group(s) a) of the invention denote(s) a mixture of monosaccharide(s) with amine group(s), one of which is glucosamine or the salts thereof of an organic or mineral acid such as hydrochloric acid, the α or β anomers thereof, the optical isomers thereof of L or D configuration, and the solvates thereof such as hydrates.

According to another form of the invention, the monosaccharide(s) with amine group(s) a) of the invention denote(s) a single monosaccharide with amine group(s), in particular glucosamine or the organic or mineral acid salts thereof or more particularly the mineral acid salts thereof such as the hydrochloric acid salt thereof, the α or β anomers thereof, the optical isomers thereof of L or D configuration, and the solvates thereof such as hydrates.

According to one particular embodiment, the monosaccharide(s) with amine group(s) a) of the invention are C₅ monosaccharides with amine group(s): pentosamines. According to this particular embodiment of the invention, the pentosamines are chosen from aldopentosamines and ketopentosamines such as xylosamine, arabinosamine, lyxosamine, ribosamine, ribulosamine and xylulosamine and also the organic or mineral acid salts thereof, the α or β anomers thereof, the optical isomers thereof of L or D configuration, and the solvates thereof such as hydrates.

According to another embodiment, the monosaccharide(s) with amine group(s) a) of the invention are C₇ monosaccharides with amine group(s): heptosamines such as aldoheptosamines and ketoheptosamines and also the organic or mineral acid salts thereof, and also the α or β anomers thereof, the optical isomers thereof of L or D configuration, and the solvates thereof such as hydrates.

According to the preferred embodiment of the invention, the monosaccharide(s) with amine group(s) a) of the invention is (are) C₆ monosaccharides: hexosamines and also the organic or mineral acid salts thereof, the α or β anomers thereof, the optical isomers thereof of L or D configuration, and the solvates thereof such as hydrates.

As examples of monosaccharides with amine group(s) a), mention may be made of C₆ monosaccharides with amine group(s) or hexosamines: aldohexosamines and ketohexosamines. According to one embodiment of the invention, the hexosamines are chosen from glucosamine, galactosamine, allosamine, altrosamine, mannosamine, gulosamine, idosamine, galactosamine and talosamine, more particularly chosen from glucosamine and galactosamine, and also the salts thereof of an organic or mineral acid such as hydrochloric acid, the α or β anomers thereof, the optical isomers thereof of L or D configuration, and the solvates thereof such as hydrates, and more preferentially glucosamine, preferably of D configuration, preferably salified with a mineral acid such as hydrochloric acid.

More preferentially, the monosaccharide(s) with amine group(s) a) of the invention are hexosamines of formula (A) and also the salts thereof of an organic or mineral acid such as hydrochloric acid, and also the α or β anomers thereof, the optical isomers thereof of L or D configuration, and the solvates thereof such as hydrates:

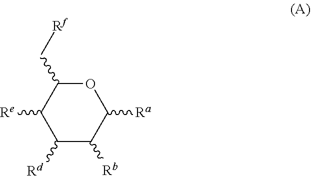

in which formula (A):
R^{a}, R^{b}, R^{d}, R^{e} and R^{f}, which are identical or different, represent i) a hydroxyl group, ii) a (C₁-C₄)alkoxy group, the alkyl group of which may be optionally substituted, especially with one or more hydroxyl groups, iii) a carboxyl group, and iv) an NR₁R₂ group, with R₁ and R₂ as defined above, in particular R₁ and R₂ are chosen from a hydrogen atom and —C(O)—R′₁ with R′₁ being as defined above; preferably, R₁ and R₂ represent i) a hydrogen atom or ii) a —C(O)—R′₁ alkylcarbonyl group with R′₁ representing a (C₁-C₄)alkyl group such as methyl; it being understood that at least one of the R^{a}, R^{b}, R^{d}, R^{e} and R^{f} radicals represents an NR₁R₂ group, preferably at least one of the R^{a}, R^{b}, R^{d}, R^{e} and R^{f} radicals represents an NR₁R₂ group and the radicals different from NR₁R₂ denote a hydroxyl group, more particularly R^{b} represents an NR₁R₂ group such as NH₂ or —N(H)—C(O)—R′₁ with R′₁ representing a (C₁-C₄)alkyl group such as methyl, more particularly NH₂, and R^{a}, R^{d}, R^{e} and R^{f} represent a hydroxyl group.

Preferably, the compounds of formula (A) are of D configuration, also referred to as D-glucopyrans. The compounds of formula (A) are particularly of β (beta) anomeric configuration.

According to one particular embodiment, the monosaccharide(s) with amine group(s) a) of the invention are chosen from the compounds of formula (A′) below and also the salts thereof of an organic or mineral acid, preferably a mineral acid such as hydrochloric acid, and also the α or β anomers thereof, the optical isomers thereof of L or D configuration, preferably D configuration, and the solvates thereof such as hydrates:

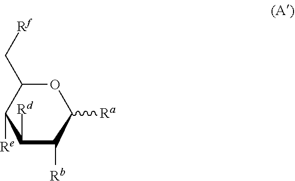

in which formula (A′) R^{a}, R^{b}, R^{d}, R^{e} and R^{f} are as defined for (A) above.

According to one preferred embodiment, the monosaccharide(s) with amine group(s) of the invention a) is (are) present in the composition (C) or in the composition (ca) in an amount of between 0.01% and 20% by weight relative to the total weight of the composition which comprises same, more particularly between 0.05% and 10%, more preferentially between 0.1% and 5% by weight relative to the total weight of the composition which comprises same.

b) Polysaccharides with Amine Group(s)

The second ingredient used in the present invention is b) one or more polysaccharides with amine group(s), and also the organic or mineral acid salts thereof, the α or β anomers thereof, the optical isomers thereof of L or D configuration, and the solvates thereof such as hydrates.

According to one preferred embodiment of the invention, the polysaccharide(s) with amine group(s) b) has (have) an average molecular weight MW of less than or equal to 400 kDa, particularly less than 200 kDa. According to one particular embodiment, the polysaccharide(s) with amine group(s) b) have a low average molecular weight MW i.e. have an MW<100 kDa, preferentially have an average MW which is <40 kDa, more preferentially have an average MW which is between 1 kDa and 30 kDa inclusive, more preferentially still have an average MW which is between 3 kDa and 28 kDa inclusive.

The polysaccharide(s) with amine group(s) b) of the invention is (are) of natural animal or plant origin, or else are derived from synthesis, hemisynthesis or biosynthesis.

According to one particular embodiment of the invention, the polysaccharide(s) with amine group(s) b) is (are) chosen from those with C₅-C₇ saccharide units and also the organic or mineral acid salts thereof, the α or β anomers thereof, the optical isomers thereof of L or D configuration, and the solvates thereof such as hydrates.

More particularly, the polysaccharide(s) with amine group(s) b) is (are) a C₆ saccharide unit with amine group(s), these polysaccharides with amine group(s) are then referred to as polyhexosamines.

According to one particular embodiment, the saccharide units of the polysaccharide with amine group(s) b) are of β (beta) anomeric configuration and/or D configuration.

According to one particular embodiment, the saccharide units of the polysaccharide with amine group(s) b) are joined to one another between the C1 carbon atoms of one saccharide unit and the C4 carbon atoms of the other saccharide unit, denoted (1-4), such as the polysaccharide with amine group(s) of formula (B) below, and also the organic or mineral acid salts thereof, the α or β anomers thereof, the optical isomers thereof of L or D configuration, and the solvates thereof such as hydrates:

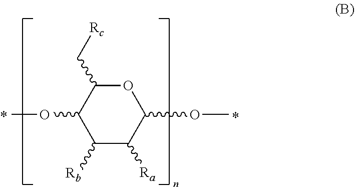

in which formula (B):
the Rₐ, R_{b}, R_{c} radicals of each saccharide unit may be identical or different;
n is an integer greater than or equal to 2, particularly between 3 and 3000 inclusive, and more particularly between 5 and 2500, preferentially between 10 and 2300;
Rₐ, R_{b}, and R_{c}, which are identical or different, represent i) a hydroxyl group, ii) a (C₁-C₄)alkoxy group, the alkyl group of which may be optionally substituted, especially with one or more hydroxyl groups, iii) a carboxyl group, and iv) an NR₁R₂ group, with R₁ and R₂ as defined above, in particular R₁ and R₂ are chosen from a hydrogen atom and —C(O)—R′₁ in which R′₁ is as defined above; preferably R₁ and R₂ represent i) a hydrogen atom or ii) —C(O)—R′₁ with R′₁ representing a (C₁-C₄)alkyl group such as methyl;
   it being understood that at least one of the Rₐ, R_{b} or R_{c} radicals of at least one saccharide unit represents an NR₁R₂ group and that at least one of the NR₁R₂ groups of at least one saccharide unit represents an NH₂ group; preferably R^{a} of at least one saccharide unit represents an NR₁R₂ group with R₁ which represents a hydrogen atom and R₂ is chosen from i) a hydrogen atom or ii) a —C(O)—R′₁ group, and R_{b} and R_{c} represent a hydroxyl group, it being understood that at least one of the NR₁R₂ groups of at least one saccharide unit represents an NH₂ group.

More particularly, the polysaccharide(s) with amine group(s) b) of the invention is (are) of formula (B₁) below, and also the organic or mineral acid salts thereof, the α or β anomers thereof, the optical isomers thereof of L or D configuration, and the solvates thereof such as hydrates:

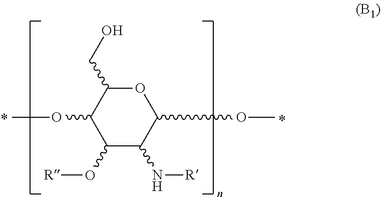

in which formula (B₁):
R′ represents a hydrogen atom or a (C₁-C₄)alkylcarbonyl group such as acetyl CH₃—C(O)—;
R″ represents a hydrogen atom or a (C₁-C₄)alkyl group optionally substituted with a carboxyl group such as —CH(CO₂H)—CH₃;
n is an integer greater than or equal to 2, particularly between 3 and 3000 inclusive, more particularly between 5 and 2500, preferentially between 10 and 2300;
   it being understood that in the polysaccharide (B₁) at least one saccharide unit bears an NH₂ amino group and at least one other saccharide unit bears at least one N(H)—R′ group with R′ representing a (C₁-C₄)alkylcarbonyl group such as acetyl CH₃—C(O)—.

Preferably, the saccharide units of formula (B) or (B₁) are of D configuration, also referred to as D-glucopyran. The units of formula (B) or (B₁) are particularly of β (beta) anomeric configuration. According to one particular embodiment, the polysaccharides of the invention are chosen from the compounds of formula (B₂) below and also the organic or mineral acid salts thereof, and the solvates thereof such as hydrates:

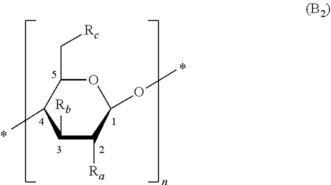

in which formula (B₂):
Rₐ, R_{b}, and R_{c}, are as defined for (B) above; and
the Rₐ, R_{b}, R_{c} radicals of each saccharide unit may be identical or different;
n is an integer greater than or equal to 2, particularly between 3 and 3000 inclusive, more particularly between 5 and 2500, preferentially between 10 and 2300;
   it being understood that in the polysaccharide (B₂) at least one of the Rₐ, R_{b} or R_{c} radicals of at least one saccharide unit represents an NR₁R₂ group and that at least one of the NR₁R₂ groups of at least one saccharide unit represents an NH₂ group; preferably at least one saccharide unit bears an R^{a} amino NH₂ group and at least one other saccharide unit bears an R^{a} group which represents —N(H)—R′ with R′ representing a (C₁-C₄)alkylcarbonyl group such as acetyl CH₃—C(O)—.

Preferentially, the polysaccharide(s) with amine group(s) of the invention b) is (are) chosen from chitin and chitosan and their derivatives, preferably chitosan.

More particularly, the polysaccharide(s) with amine group(s) of the invention b) is (are) chosen from those of formula (B₃) below, and also the organic or mineral acid salts thereof, and the solvates thereof such as hydrates:

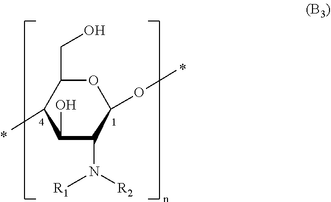

in which formula (B₃):
R₁ and R₂ are as defined in the formula (B), (B₁) or (B₂); and
n is an integer greater than or equal to 2, particularly between 3 and 3000 inclusive, more particularly between 5 and 2500, preferentially between 10 and 2300;
   it being understood that in the polysaccharide b) of formula (B₃) at least one saccharide unit bears an NH₂ amino group and at least one other saccharide unit bears an N(H)—R′ group with R′ representing a (C₁-C₄)alkylcarbonyl group such as acetyl CH₃—C(O)—.

More particularly, the polysaccharide(s) with amine group(s) of the invention b) is (are) chosen from chitosans of formula (B₄) below, and also the organic or mineral acid salts thereof, and the solvates thereof such as hydrates:

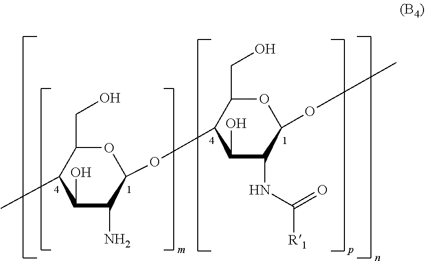

in which formula (B₄):
R′₁ representing a (C₁-C₄)alkyl group such as methyl; and
n is an integer greater than or equal to 2, particularly between 3 and 3000 inclusive, more particularly between 5 and 2500, preferentially between 10 and 2300;
P is greater than 0 and ranges up to 0.5, preferably from 0.05 to 0.3, and better still from 0.1 to 0.20 such as 0.15 with m+p being equal to 1;
   it being understood that in the chitosan at least one saccharide unit bears an NH₂ amino group and at least one other saccharide unit bears an N(H)—R′₁ group with R′ representing a (C₁-C₄)alkylcarbonyl group such as acetyl CH₃—C(O)—.
   For example, when m=0.7, p=0.3 this means that 70% of the amine groups are free (unsubstituted) and 30% of the amino groups are N-alkyl(C₁-C₄)carbonyl groups, in particular N-acetyl groups, corresponding to the chitosan of formula:

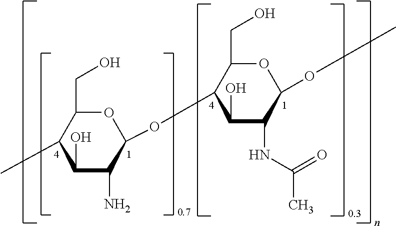

with n as defined above.

Particularly, the polysaccharide(s) with amine group(s) of the invention b) is (are) chosen from chitosans, salified using organic acid, preferentially using monocarboxylic acid of formula (I) as defined above or polycarboxylic acid of formula (II) as defined above, more preferentially still salified using carboxylic acid of formula (I) such as lactic acid.

According to one particular form of the invention, the polysaccharide(s) with amine group(s) b) of the invention denote(s) a mixture of polysaccharide(s) with amine group(s), one of which is a chitosan or the organic or mineral acid salts thereof, preferably the salts thereof of an organic acid such as lactic acid, the α or β anomers thereof, the optical isomers thereof of L or D configuration, and the solvates thereof such as hydrates.

According to another form of the invention, the polysaccharide(s) with amine group(s) b) of the invention denote(s) a single polysaccharide with amine group(s), in particular a mixture of chitosan or the organic or mineral salts thereof or more particularly the organic acid salts thereof such as the lactic acid salt thereof, the α or β anomers thereof, the optical isomers thereof of L or D configuration, and the solvates thereof such as hydrates.

According to another form of the invention, the polysaccharide(s) with amine group(s) b) of the invention denote(s) a single polysaccharide with amine group(s), in particular a chitosan or the organic or mineral acid salts thereof or more particularly the organic acid salts thereof such as the lactic acid salt thereof, the α or β anomers thereof, the optical isomers thereof of L or D configuration, and the solvates thereof such as hydrates.

According to one preferred embodiment, the polysaccharide(s) with amine group(s) of the invention b) in particular of formula (B), (B₁), (B₂), (B₃) or (B₄) as defined above is (are) present in the composition (C) or in the composition (cb) in an amount of between 0.01% and 20% by weight relative to the total weight of the composition which comprises same, more particularly between 0.05% and 10%, more preferentially between 0.1% and 5% by weight relative to the total weight of the composition which comprises same.

The Compositions

The composition of the invention (C) and the composition(s) (ca) and/or (cb) used in the invention or the process of the invention comprising the ingredients a) and/or b) as defined above are cosmetic, i.e. contain a physiologically acceptable medium, that is to say that is compatible with human keratin materials such as the skin (of the body, face, around the eyes or the scalp), the hair, the eyelashes, the eyebrows, body hair, the nails or the lips.

The physiologically acceptable medium of the composition(s) (C), (ca), (cb) used in the process according to the invention is advantageously an aqueous medium. It may for example contain water or a mixture of water and at least one cosmetically acceptable organic solvent. Examples of cosmetically acceptable organic solvents that may be mentioned include C₂-04 lower alcohols, such as ethanol and isopropanol; polyols, especially those containing from 2 to 6 carbon atoms, for instance glycerol, propylene glycol, butylene glycol, pentylene glycol, hexylene glycol, dipropylene glycol or diethylene glycol; polyol ethers, for instance 2-butoxyethanol, propylene glycol monomethyl ether and diethylene glycol monomethyl ether or monoethyl ether; and mixtures thereof.

Preferably, the cosmetic composition(s) comprise from 50% to 99.8% by weight of water, relative to the weight of the composition(s).

The composition(s) used according to the invention may also contain one or more cosmetic additives usually used in cosmetics chosen from nonionic, anionic, cationic and amphoteric surfactants, vitamins and provitamins, including panthenol, fillers, opacifiers, sequestrants, film-forming polymers, thickeners, antioxidants, antifoams, moisturizers, emollients, penetrants, fragrances and preserving agents.

The composition(s) (C), (ca), (cb) used according to the invention may be in any presentation form conventionally used for hair application.

pH of the Compositions:

The pH values of the composition(s) (C), (ca), (cb) of the invention may be adjusted with an organic or mineral acid, or with an alkaline agent chosen from mineral, organic or hybrid alkaline agents or mixtures thereof.

According to one particular embodiment of the invention, the composition (ca) which comprises a) one or more monosaccharides with amine group(s) as defined above, in particular glucosamine, is at an acid pH, i.e. a pH below 7, in particular below or equal to 6, more particularly at a pH between 2 and 6 inclusive, preferentially at a pH between 2.5 and 5.5, more preferentially between 3.5 and 5 such as 4.5. According to one preferred embodiment of the invention, the monosaccharide(s) with amine group(s) a) are in the presence of one or more mineral acids, more particularly hydrochloric acid.

The pH values may be adjusted with c) one or more organic or mineral acids as defined above in “organic or mineral acid salt”.

According to one preferred embodiment of the invention, the composition (cb) which comprises b) one or more polysaccharides with amine group(s) as defined above, in particular of formula (B), (B₁), (B₂) or (B₃) or (B₄) as defined above such as chitosan, is at an acid pH, i.e. a pH below 7, in particular below or equal to 6, more particularly at a pH between 2 and 6 inclusive, preferentially at a pH between 2.5 and 5.5, more preferentially between 3.5 and 5 such as 4.5. According to one particular embodiment of the invention, the polysaccharide(s) with amine group(s) b) are in the presence of one or more organic acids, preferentially monocarboxylic acid(s) of formula (I) or polycarboxylic acid(s) of formula (II) as defined above, more preferentially still one or more carboxylic acids of formula (I) such as lactic acid.

According to a particular embodiment of the invention, the composition (C) comprises:
a) one or more monosaccharides with amine group(s) as defined above, in particular glucosamine, in the presence of one or more mineral acids, more particularly hydrochloric acid, and
b) one or more polysaccharides with amine group(s) as defined above, in particular of formula (B), (B₁), (B₂) or (B₃) or (B₄) as defined above, especially chitosan, in particular chitosan having an average molecular weight of less than or equal to 400 kDa, in the presence of one or more organic acids, more particularly lactic acid;
and optionally c) one or more mineral or organic acids preferably different from the following acids: aspartic acid, glutamic acid, gluconic acid, pyrrolidonecarboxylic acid, 100 OE and 500 OE ethoxylated stearic acid, linoleic acid, succinic acid,
   and is at an acid pH, i.e. a pH below 7, in particular below or equal to 6, more particularly at a pH between 2 and 6 inclusive, preferentially at a pH between 2.5 and 5.5, more preferentially between 3.5 and 5 such as 4.5.

According to another particular embodiment of the invention, the composition (C) comprises:
a) one or more monosaccharides with amine group(s) as defined above, in particular glucosamine, in the absence of mineral or organic acids, and
b) one or more polysaccharides with amine group(s) as defined above, in particular of formula (B), (B₁), (B₂) or (B₃) or (B₄) as defined above, especially chitosan, in the presence of one or more organic acids, more particularly lactic acid,
and optionally c) one or more mineral or organic acids preferably different from the following acids: aspartic acid, glutamic acid, gluconic acid, pyrrolidonecarboxylic acid, 100 OE and 500 OE ethoxylated stearic acid, linoleic acid, succinic acid,
   and is at an acid pH, i.e. a pH below 7, in particular below or equal to 6, more particularly at a pH between 2 and 6 inclusive, preferentially at a pH between 2.5 and 5.5, more preferentially between 3.5 and 5 such as 4.5.

According to a particular embodiment of the invention, the composition (C) comprises:
a) one or more monosaccharides with amine group(s) as defined above, in particular glucosamine, in the presence of one or more mineral acids, more particularly hydrochloric acid, and
b) one or more polysaccharides with amine group(s) as defined above, in particular of formula (B), (B₁), (B₂) or (B₃) or (B₄) as defined above, especially chitosan, in particular chitosan having an average molecular weight of less than or equal to 400 kDa, in the absence of organic or mineral acid(s),
and optionally c) one or more mineral or organic acids preferably different from the following acids: aspartic acid, glutamic acid, gluconic acid, pyrrolidonecarboxylic acid, 100 OE and 500 OE ethoxylated stearic acid, linoleic acid, succinic acid,
   and is at an acid pH, i.e. a pH below 7, in particular below or equal to 6, more particularly at a pH between 2 and 6 inclusive, preferentially at a pH between 2.5 and 5.5, more preferentially between 3.5 and 5 such as 4.5.

According to a particular embodiment of the invention, the composition (C) comprises:
a) one or more monosaccharides with amine group(s) as defined above, in particular glucosamine, in the absence of mineral acid(s),
b) one or more polysaccharides with amine group(s) as defined above, in particular of formula (B), (B₁), (B₂) or (B₃) or (B₄) as defined above, especially chitosan, in particular chitosan having an average molecular weight of less than or equal to 400 kDa, in the absence of organic or mineral acid(s),
and c) one or more mineral or organic acids different from the following acids: aspartic acid, glutamic acid, gluconic acid, pyrrolidonecarboxylic acid, 100 OE and 500 OE ethoxylated stearic acid, linoleic acid, succinic acid, said acid c) preferably being chosen from hydrochloric acid, organic monocarboxylic acids of formula (I) as defined above and in particular lactic acid, citric acid, polycarboxylic acids of formula (II) as defined above and in particular fumaric or maleic acid,
   and is at an acid pH, i.e. a pH below 7, in particular below or equal to 6, more particularly at a pH between 2 and 6 inclusive, preferentially at a pH between 2.5 and 5.5, more preferentially between 3.5 and 5 such as 4.5.

The pH values may also be adjusted with one or more mineral alkaline agents which are preferably chosen from aqueous ammonia, alkali metal carbonates or bicarbonates such as sodium or potassium carbonates and sodium or potassium bicarbonates, sodium hydroxide or potassium hydroxide, or mixtures thereof.

The pH values may also be adjusted with one or more organic alkaline agents which are organic amines i.e they contain at least one substituted or unsubstituted amino group. The organic alkaline agent(s) is (are) more preferentially chosen from organic amines with a pK_{b} at 25° C. of less than 12, and preferably of less than 10, even more advantageously of less than 6. It should be noted that it is the pK_{b} corresponding to the function of highest basicity.

The pH values may also be adjusted with one or more hybrid compounds. Hybrid compounds that may be mentioned include the salts of the amines mentioned previously with acids such as carbonic acid or hydrochloric acid.

The organic alkaline agent(s) are chosen, for example, from alkanolamines, oxyethylenated and/or oxypropylenated ethylenediamines, amino acids and the compounds of formula (III) below:

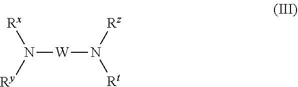

in which formula (III):
W is a divalent C₁-C₆ alkylene radical optionally substituted with a hydroxyl group or a C₁-C₆ alkyl radical, and/or optionally interrupted with one or more heteroatoms such as oxygen or NR^{u};
R^{x}, R^{y}, R^{x}, R^{t} and R^{u}, which may be identical or different, represent a hydrogen atom or a C₁-C₆ alkyl, C₁-C₆ hydroxyalkyl or C₁-C₆ aminoalkyl radical.

The Composition of the Invention

According to a particular embodiment of the invention, the composition (C) is the composition (C′) at acid pH and comprises:
a) one or more salified monosaccharides with amine group(s) as defined above;
b) one or more salified polysaccharides with amine group(s) as defined above, preferably having an average molecular weight of less than or equal to 400 kDa, and optionally c) one or more mineral or organic acids preferably different from the following acids: aspartic acid, glutamic acid, gluconic acid, pyrrolidonecarboxylic acid, 100 OE and 500 OE ethoxylated stearic acid, linoleic acid, succinic acid.

According to another particular embodiment of the invention, the composition (C) is the composition (C′) at acid pH and comprises:
a) one or more non-salified monosaccharides with amine group(s) as defined above; and
b) one or more salified polysaccharides with amine group(s) as defined above, preferably having an average molecular weight of less than or equal to 400 kDa, and optionally c) one or more mineral or organic acids preferably different from the following acids: aspartic acid, glutamic acid, gluconic acid, pyrrolidonecarboxylic acid, 100 OE and 500 OE ethoxylated stearic acid, linoleic acid, succinic acid.

According to another particular embodiment of the invention, the composition (C) is the composition (C′″) at acid pH and comprises:
a) one or more salified monosaccharides with amine group(s) as defined above; and
b) one or more non-salified polysaccharides with amine group(s) as defined above, preferably having an average molecular weight of less than or equal to 400 kDa,
and optionally c) one or more mineral or organic acids preferably different from the following acids: aspartic acid, glutamic acid, gluconic acid, pyrrolidonecarboxylic acid, 100 OE and 500 OE ethoxylated stearic acid, linoleic acid, succinic acid.

According to yet another particular embodiment of the invention, the composition (C) is the composition (C″″) at acid pH and comprises:
a) one or more non-salified monosaccharides with amine group(s) as defined above;
b) one or more non-salified polysaccharides with amine group(s) as defined above, preferably having a low average molecular weight of less than or equal to 400 kDa, and
c) one or more organic or mineral acids as defined above; preferably different from the following acids: aspartic acid, glutamic acid, gluconic acid, pyrrolidonecarboxylic acid, 100 OE and 500 OE ethoxylated stearic acid, linoleic acid, succinic acid.

Process for Treating Keratin Fibres

The process for treating keratin fibres, in particular human keratin fibres such as the hair, comprises:
(i) a step consisting in applying, to said fibres, a) one or more monosaccharides with amine group(s);
(ii) a step consisting in applying, to said fibres, b) one or more polysaccharides with amine group(s), preferably having an average molecular weight of less than or equal to 400 kDa;
(ii′) optionally a drying step;
(iii) then a step of heat treatment at a temperature above or equal to 80° C., in particular at a temperature between 100° C. and 250° C., preferably with a hair iron;
   it being understood that the steps (i) and (ii) may be carried out simultaneously, or sequentially, preferably the steps (i) and (ii) are carried out simultaneously and that the drying step (ii′), when it is present, precedes the heat treatment step and follows the steps (i) and (ii).

Another subject of the invention is a process for treating keratin fibres, especially human keratin fibres, in particular the hair, comprising:
(i) a step consisting in applying, to said fibres, a cosmetic composition (ca) containing a) one or more monosaccharides with amine group(s);
(ii) a step consisting in applying, to said fibres, a cosmetic composition (cb) containing b) one or more polysaccharides with amine group(s), preferably having an average molecular weight of less than or equal to 400 kDa;
(ii′) optionally a drying step;
(iii) then a step of heat treatment at a temperature above or equal to 80° C., in particular at a temperature between 100° C. and 250° C., preferably with a hair iron;
   it being understood that the steps (i) and (ii) may be carried out simultaneously, or sequentially, preferably the steps (i) and (ii) are carried out simultaneously and that the drying step (ii′), when it is present, precedes the heat treatment step and follows the steps (i) and (ii).

According to a particular embodiment of the process of the invention, steps i) and ii) are carried out simultaneously, or else by applying to the keratin fibres, during a first step, a composition comprising (a) one or more monosaccharides with amine group(s) a) as defined above; and one or more polysaccharides with amine group(s) b) as defined above, i.e. by applying, during a first step, a composition (C) as defined above.

According to a particular embodiment of the invention and the composition (C), the monosaccharide(s) with amine group(s) a) as defined above; and the polysaccharide(s) with amine group(s) b) as defined above are each found in said composition in an amount of between 0.01% and 20% by weight relative to the total weight of the composition, more particularly between 0.05% and 10%, more preferentially between 0.1% and 5%, more particularly between 0.1% and 5% by weight relative to the total weight of the composition (C) comprising same.

Preferably, the composition (ca) applied to said fibres is acidic and comprises one or more organic or mineral acids preferably c) as defined above.

More preferentially, the process of the invention comprises a first step that consists in applying the composition (C) as defined above, optionally followed by a drying step, then a step of heat treatment at a temperature above 80° C., in particular at a temperature between 100° C. and 250° C., preferably with a hair iron. According to a first advantageous variant, the composition (C) is the composition (C′) as defined above. According to another advantageous variant, the composition (C) represents a composition (C″) as defined above. According to another advantageous variant, the composition (C) represents a composition (C′″) as defined above. According to yet another advantageous variant, the composition (C) represents the composition (C″″) as defined above.

According to a particular variant of the invention, the process of the invention and the use of the invention do not use oxidized polysaccharide, more generally the process of the invention and the use of the invention do not use a compound comprising at least one aldehyde group. According to one preferred embodiment of the invention, the composition(s) (cb), (C), (C′), (C″), (C′″), or (C″″) as defined above does not (do not) contain oxidized polysaccharide and more generally the composition(s) (cb), (C), (C′), (C″), (C′″), or (C″″) does not (do not) contain a compound comprising at least one aldehyde group.

According to a particular embodiment, the process of the invention is carried out after permanent waving, smoothing, dyeing and/or bleaching of the keratin fibres, in particular human keratin fibres such as the hair.

The process according to invention may also comprise an additional step (ii′) of drying the keratin fibres after the application of the monosaccharide(s) with amine group(s) a) as defined above and/or of the polysaccharide(s) with amine group(s) b) as defined above or of the cosmetic compositions containing same and before the step of heating the keratin fibres carried out at a temperature of at least 80° C.

Preferably, the process of the invention carries out a step (ii′) of drying the keratin fibres, in particular human keratin fibres such as the hair before the heat treatment step (iii). The drying step is in particular performed using a hairdryer, a hood, or else with a towel, an absorbent or by drying naturally. The drying step is advantageously carried out at a temperature ranging from 20° C. to 70° C.

The keratin fibre heat treatment or heating step (iii) of the process of the invention is preferably carried out at a temperature between 80° C. and 250° C., it being understood that the temperature is that of the heating means, in particular of the hair iron, and not the temperature of the keratin fibres.

According to one preferred form of the invention, the heat treatment step iii) is carried out on dry keratin fibres.

According to a more particular embodiment of the invention and the process for treating keratin fibres, the fibre heat treatment step or heating step iii) is carried out at a temperature of at least 100° C., in particular at a temperature of between 100° C. and 250° C. inclusive. Preferably, the step of heating the keratin fibres is carried out at a temperature ranging from 150° C. to 220° C., preferably ranging from 160° C. to 220° C., preferentially ranging from 160° C. to 200° C., especially ranging from 130° C. to 190° C. It is understood that the heating temperatures are the temperatures of the heating means, in particular of the hair iron when a hair iron is used, and not the temperature of the keratin fibres. This heat treatment or heating step (iii) is advantageously carried out using a hair iron.

For the purposes of the present invention, the term “hair iron” means a device for heating keratin fibres by placing said fibres in contact with the heating device.

The end of the hair iron which comes into contact with the keratin fibres generally has two surfaces. These two surfaces may be made of metal or of ceramic. In particular, these two surfaces may be smooth or crimped or curved.

The heating step may be carried out by means of a straightening iron, a curling iron, a crimping iron or a steam iron such as the Steampod. Preferably, the heating step is carried out using a straightening iron.

As examples of hair irons that may be used in the process according to the invention, mention may be made of any type of flat hair iron, and in particular, in a nonlimiting manner, those described in patents U.S. Pat. Nos. 5,957,140 and 5,046,516.

The hair iron may be applied by successive separate strokes lasting a few seconds or by gradual movement or sliding along the locks of keratin fibres, especially of hair.

Preferably, the hair iron is applied in the process according to the invention by a continuous movement from the root to the end of the keratin fibres, especially human keratin fibres such as the hair, in one or more passes, in particular in one to twenty passes. Each pass of the hair iron may last from 2 seconds to 1 minute for a length of the treated keratin fibres of 30 cm±3 cm.

Preferably, the step of heating the keratin fibres is performed for a time that may range from 100 milliseconds to 30 minutes, and preferentially from 1 second to 15 minutes, better still from 2 seconds to 10 minutes, better still from 3 seconds to 5 minutes and even better still from 4 seconds to 1 minute.

After the heat treatment step (iii) of the process, the keratin fibres may be optionally rinsed with water or washed with a shampoo. The keratin fibres are subsequently optionally dried using a hairdryer or a hood or naturally.

According to one embodiment, the process according to the invention is performed on keratin fibres, in particular human keratin fibres, such as the hair, especially that are natural, especially natural hair.

According to another embodiment, the process according to the invention is carried out on keratin fibres, especially human keratin fibres, such as the hair, which is damaged. As indicated previously, the term “damaged hair” means dry, coarse, brittle, split and/or limp hair.

According to another embodiment, the treatment process according to the invention is preferably carried out on keratin fibres, especially human keratin fibres such as the hair, which are sensitized, such as bleached, artificially dyed, relaxed or permanent-waved fibres. The process according to the invention may be performed on keratin fibres, especially human keratin fibres such as the hair, which are dry or wet. Preferentially, the process is performed on dry keratin fibres, especially dry hair.

The cosmetic composition(s) used according to the invention are advantageously applied to the keratin fibres in an amount ranging from 0.01 to 10 grams and preferably from 0.1 to 5 grams, more preferably from 0.5 to 3 grams of composition per gram of keratin fibres.

After application of the cosmetic composition to the keratin fibres, they may be wrung out to remove the excess composition.

After application to the keratin fibres of the ingredient a) and/or the ingredient b) as defined above, or of a cosmetic composition containing the same, and before carrying out the keratin fibre heat treatment or heating step (iii), the ingredient(s) a) as defined above and/or the ingredient(s) b) as defined above or the composition(s) containing the same may be left on for a time ranging from 1 to 60 minutes, preferably ranging from 2 to 50 minutes and preferentially ranging from 5 to 45 minutes. The leave-on period may take place at a temperature ranging from 15° C. to 45° C., preferably at room temperature (25° C.). Preferably, the process of the invention carries out the steps (i) and (ii) simultaneously, the leave-on period of the ingredients (a) and (b) is then between 1 second and 60 minutes. According to one embodiment, the process of the invention comprises:
(i) a first step consisting in applying, to said fibres, a) one or more monosaccharides with amine group(s) as defined above, preferably in salified form;
   then optionally a drying step ii′),
   then
(ii) a second step consisting in applying, to said fibres, b) one or more polysaccharides with amine group(s) as defined above, preferably that are salified, particularly having an average molecular weight of less than or equal to 400 kDa,
then optionally a drying step ii′),
   then
(iii) a step of heat treatment at a temperature of at least 80° C., in particular at a temperature between 80° C. and 250° C.;
   it being understood that the ingredient(s) a) as defined above and the ingredient(s) b) as defined above are present in different cosmetic compositions. They are therefore applied separately, i.e. sequentially, to the keratin fibres.

According to another particular variant of the invention, the process of the invention comprises:
(i) a first step consisting in applying, to said fibres, b) one or more polysaccharides with amine group(s) as defined above, preferably in salified form, particularly having an average molecular weight of less than or equal to 400 kDa, then optionally a drying step ii′),
then
(ii) a second step consisting in applying, to said fibres, a) one or more monosaccharides with amine group(s) as defined above, preferably in salified form; then optionally a drying step ii′),
then
(iii) a step of heat treatment at a temperature of at least 80° C., in particular at a temperature between 80° C. and 250° C.;
   it being understood that the ingredient(s) a) as defined above and the ingredient(s) b) as defined above are present in different cosmetic compositions. They are therefore applied separately, i.e. sequentially, to the keratin fibres.

According to one particular embodiment, the process of the invention sequentially carries out a first step (i) that consists in applying, to keratin fibres, the monosaccharide(s) with amine group(s) a) as defined above or a composition (ca) containing the same, which is/are left on for a time of between 1 min and 30 min inclusive, then a second step (ii) that consists in applying b) the polysaccharide(s) with amine group(s) b) as defined above or a composition (cb) containing same, which is/are left on for between 1 second and 30 minutes, then (ii′) said fibres are dried, followed by a heat treatment step (iii).

According to another particular embodiment, the process of the invention carries out a first step (i) that consists in applying, to the keratin fibres, the monosaccharide(s) with amine group(s) a) as defined above simultaneously with the polysaccharide(s) with amine group(s) b) as defined above, which are left on for a time of between 1 min and 60 minutes inclusive, then (ii′) said fibres are dried, followed by a heat treatment step (iii).

According to this embodiment, the process of the invention carries out a first step (i) that consists in applying, to the keratin fibres, a composition (C), (C′), (C′), (C″), or (C″″) containing the monosaccharide(s) with amine group(s) a) as defined above and the polysaccharide(s) with amine group(s) b) as defined above, which are left on for a time of between 1 min and 60 min inclusive, then (ii′) said fibres are dried, followed by a heat treatment step (iii).

According to a particular embodiment of the process of the invention, the step (i) of applying the monosaccharide(s) with amine group(s) a) uses a composition (ca) comprising one or more monosaccharides with amine group(s) a) as defined above in a content ranging between 0.01% and 20% by weight relative to the total weight of the composition which comprises same, more particularly between 0.05% and 10%, more preferentially between 0.1% and 5%, more preferentially still between 0.1% and 1.5% by weight relative to the total weight of the composition comprising same.

According to a particular embodiment of the invention, the step (ii) of the process of applying the polysaccharide(s) with amine group(s) b) uses a composition (cb) comprising one or more polysaccharides with amine group(s) b) as defined above in an amount of between 0.01% and 20% by weight relative to the total weight of the composition, more particularly between 0.05% and 10%, more preferentially between 0.1% and 5%, more particularly between 0.1% and 5% by weight relative to the total weight of the composition comprising same.

According to an embodiment of the process according to the invention, the ingredients a) and b) as defined above are present in the same cosmetic composition (C), (C′), (C″), (C′″), or (C″″). They are therefore applied simultaneously to the keratin fibres. In this case, the preferred amounts are as follows:
for the monosaccharide(s) with amine group(s) a) as defined above, the amount is between 0.05% and 15% by weight, relative to the total weight of the composition, preferably between 0.1% and 10% by weight, more preferentially between 0.2% and 6% by weight relative to the total weight of the composition;
for the polysaccharide(s) with amine group(s) b) as defined above, the specific amount is between 0.01% and 10% by weight, relative to the total weight of the composition, more particularly between 0.1% and 5% by weight relative to the total weight of the composition, preferentially an amount of between 0.01% and 1.5%, more preferentially between 0.1% and 1.1% by weight relative to the total weight of the composition.

The treatment process according to the invention may be carried out before, during and/or after an additional process of cosmetic treatment of the keratin fibres, such as a process for temporarily shaping (shaping with curlers, a crimping iron or a straightening iron) or a process for durably shaping (permanent-waving or relaxing) the keratin fibres.

The treatment process may be performed as a pre-treatment to a dyeing or relaxing process and/or a permanent-waving process so as to cosmetically protect the keratin fibres against these treatments. In other words, this process is performed to preserve the cosmetic properties of the keratin fibres before a dyeing, relaxing and/or a permanent-waving cosmetic treatment process.

The treatment process according to the invention may be performed as a post-treatment to a bleaching, artificial dyeing or relaxing process and/or a permanent-waving process so as to repair the damaged keratin fibres. The keratin fibres may be damaged by overexposure to the environment (for example natural long hair may be photodegraded, or degraded mechanically by repeated brushing or combing).

In particular, the treatment process according to the invention may be performed on damaged keratin fibres.

In other words, the treatment process according to the invention is preferably carried out on sensitized keratin fibres such as bleached, dyed, relaxed or permanent-waved fibres.

In particular, the treatment process may be carried out before a bleaching, dyeing or relaxing process and/or a permanent-waving process on keratin fibres.

Kit

Another subject of the invention is a multi-compartment kit comprising:
a first cosmetic composition comprising a) at least one monosaccharide with amine group(s) as defined above and optionally c) one or more organic or mineral acids as defined above;
a second cosmetic composition comprising b) at least one polysaccharide with amine group(s) as defined above and optionally c) one or more organic or mineral acids as defined above; and
a device for heating the keratin fibres at a temperature of at least 80° C., preferably at a temperature of at least 100° C., in particular at a temperature between 100° C. and 250° C. inclusive. Preferably, the step of heating the keratin fibres is carried out at a temperature ranging from 150° C. to 220° C., preferably ranging from 160° C. to 220° C., preferentially ranging from 160° C. to 200° C., especially ranging from 130° C. to 190° C.;
   the first and second compositions each being packaged in a separate packaging assembly. The contents of each compartment of the kit may be released either sequentially or simultaneously.

The composition packaging assembly is, in a known manner, any packaging that is suitable for storing cosmetic compositions (in particular a bottle, tube, spray bottle or aerosol bottle).

Such a kit allows the process for treating keratin fibres according to the invention to be performed.

The examples that follow are given as illustrations of the present invention.

The amounts indicated in the examples are expressed as weight percentages.

### EXAMPLES

The following compositions were prepared, and the results of the number of fibres is given for each composition.

| | | No. of |
|---|---|---|
| | | fibres |
| | | repaired |
| Composition | Details of the composition | (/5)** |
| 1 | Glucosamine hydrochloride (“G”) at | 0/5 |
| comparative | 1% in water, spontaneous pH of 4.8 | |
| 2 | G at 5% in water, spontaneous | 0/5 |
| comparative | pH of 4.17 | |
| 3 | Chitosan (“C”) at 1% in water* | 0/5 |
| comparative | MW 310-375K, η = 800-2000 Cp | |
| 4 | C at 1% in water* | 0/5 |
| comparative | MW approx 5000, η <100 Cp | |
| 5 | GC (G at 1% + C at 1%) in water** | 5/5 |
| invention | with C: MW 310-375K, | |
| | η = 800-2000 Cp | |
| 6 | GC (G at 1% + C at 1%) in water** | 5/5 |
| invention | with C: MW approx 5000, η <100 Cp | |
| 7 | GC (G at 1% + C at 1%) in water** | 5/5 |
| invention | with C: MW approx 28K, η <100 Cp | |
| 8 | GC (G at 5% + C at 1%) in water** | 5/5 |
| invention | with C: MW approx 5000, η <100 Cp | |

*solubilize by addition of lactic acid up to pH = 4.5 (±0.5)
**the chitosan composition is adjusted to pH 4.5 (±0.5) with lactic acid then the glucosamine hydrochloride is added.
***Split-end hair sold by IHIP (Glendale, NY 11385, USA)

The evaluation of the repair of the keratin fibres was carried out using a magnifying optical microscope (Model 175 X optical “MightyScope 1.3M Digital Microscope” sold by Aven Inc, USA—aveninc.com).

Each fibre to be evaluated that is selected for study is a natural fibre and is naturally split into two longitudinally at the end.

The end of each fibre is observed before treatment and after treatment using the microscope. The treatment of the fibres consists in soaking each fibre in the composition for 30 seconds, then each fibre is left suspended for air drying, then is subjected to a heat treatment 3 times successively with a straightening iron at 190° C. (Babyliss PRO, EP Technology 5.0 sold by Babyliss).

Each before/after treatment comparison is repeated 5 times on different fibres split at their end longitudinally.

After treatment, it is evaluated using the optical microscope whether the fibre is repaired, i.e. if the two parts of the end of the fibre are joined, the result=1, if they are not joined, the result=0.

These results show that the combination of the ingredients a) and b) followed by a heat treatment results in a repair of the fibre, unlike the ingredients a) or b) taken alone.

1.-22. (canceled)

23. A process for treating keratin fibers, comprising:
(i) applying to the fibers a composition a) comprising at least one monosaccharide with at least one amine group;
(ii) applying to the fibers a composition b) comprising at least one polysaccharide with at least one amine group;
(ii′) optionally a drying step;
(iii) then a step of heat treatment at a temperature above or equal to 80° C., with a hair iron;
wherein steps (i) and (ii) may be carried out simultaneously or sequentially.

24. The process according to claim 23, wherein the at least one monosaccharide with at least one amine group is chosen from hexosamines of formula (A), organic or mineral acid salts thereof, α or β anomers thereof, optical isomers thereof of L or D configuration, or solvates thereof: 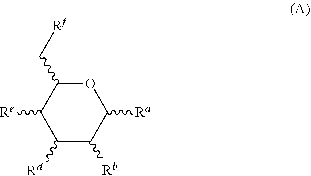
wherein:
R^{a}, R^{b}, R^{d}, R^{e}, and R^{f}, which are identical or different, represent i) a hydroxyl group, ii) a (C₁-C₄)alkoxy group, wherein the alkyl group is optionally substituted, iii) a carboxyl group, or iv) an NR₁R₂ group, wherein R₁ and R₂ represent i) a hydrogen atom or ii) a —C(O)—R′₁ alkylcarbonyl group wherein is a (C₁-C₄)alkyl group;
wherein at least one of the R^{a}, R^{b}, R^{d}, R^{e}, and R^{f} radicals represents an NR₁R₂ group.

25. The process according to claim 23, wherein the at least one monosaccharide with at least one amine group is chosen from the compounds of formula (A′) below, organic or mineral acid salts thereof, α or β anomers thereof, optical isomers thereof of L or D configuration, or solvates thereof: 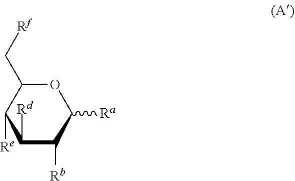
wherein
R^{a}, R^{b}, R^{d}, R^{e}, and R^{f}, which are identical or different, represent i) a hydroxyl group, ii) a (C₁-C₄)alkoxy group, wherein the alkyl group is optionally substituted, iii) a carboxyl group, or iv) an NR₁R₂ group, wherein R₁ and R₂ represent i) a hydrogen atom or ii) a —C(O)—R′₁ alkylcarbonyl group wherein is a (C₁-C₄)alkyl group;
wherein at least one of the R^{a}, R^{b}, R^{d}, R^{e}, and R^{f} radicals represents an NR₁R₂ group.

26. The process according to claim 23, wherein the at least one monosaccharide with at least one amine group is present in the composition a) in an amount of between 0.01% and 20% by weight relative to the total weight of the composition.

27. The process according to claim 23, wherein the composition a) comprising the at least one monosaccharide with at least one amine group has an acidic pH.

28. The process according to claim 23, wherein the at least one polysaccharide comprising at least one amine group is chosen from those of formula (B) below, organic or mineral acid salts thereof, α or β anomers thereof, optical isomers thereof of L or D configuration, or solvates thereof: 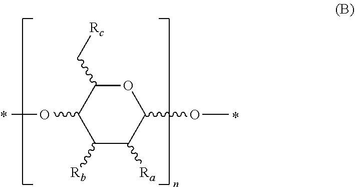
wherein:
Rₐ, R_{b}, R_{c} of each saccharide unit may be identical or different;
n is an integer greater than or equal to 2;
Rₐ, R_{b}, and R_{c}, which are identical or different, represent i) a hydroxyl group, ii) a (C₁-C₄)alkoxy group, wherein the alkyl group is optionally substituted, iii) a carboxyl group, or iv) an NR₁R₂ group, wherein R₁ and R₂ are chosen from a hydrogen atom and —C(O)—R′₁;
wherein at least one of the Rₐ, R_{b}, or R_{c} radicals represents an NR₁R₂ group wherein at least one of the NR₁R₂ groups represents an NH₂ group.

29. The process according to claim 23, wherein the at least one polysaccharide with at least one amine group is chosen from compounds of formula (B₁) below, organic or mineral acid salts thereof, α or β anomers thereof, optical isomers thereof of L or D configuration, or solvates thereof: 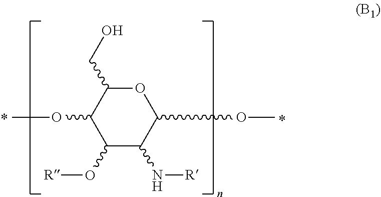
wherein:
R′ represents a hydrogen atom or a (C₁-C₄)alkylcarbonyl group;
R″ represents a hydrogen atom or a (C₁-C₄)alkyl group optionally substituted with a carboxyl group;
n is an integer greater than or equal to 2;
wherein at least one of the Rₐ, R_{b} or R_{c} radicals of at least one saccharide unit represents an NR₁R₂ group and at least one of the NR₁R₂ groups of at least one saccharide unit represents an NH₂ group.

30. The process according to claim 23, wherein the at least one polysaccharide with at least one amine group is chosen from compounds of formula (B₃) below, organic or mineral acid salts thereof, or solvates thereof: 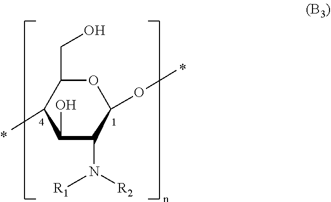
wherein:
R₁ and R₂ are chosen from a hydrogen atom and —C(O)—R′₁; and
n is an integer greater than or equal to 2;
wherein in the polysaccharide at least one saccharide unit bears an NH₂ amino group and at least one other saccharide unit bears an N(H)—R′ group with R′ representing a (C₁-C₄)alkylcarbonyl group.

31. The process according to claim 23, wherein the at least one polysaccharide with at least one amine group is chosen from chitosans of formula (B₄) below, organic or mineral acid salts thereof, or solvates thereof: 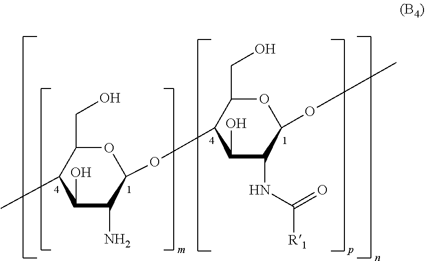
wherein:
R′₁ representing a (C₁-C₄)alkyl group; and
p is greater than 0 and ranges up to 0.5, with m+p being equal to 1;
n is an integer greater than or equal to 2;
wherein in the chitosan at least one saccharide unit bears an NH₂ amino group and at least one other saccharide unit bears an N(H)—R′₁ group with R′ representing a (C₁-C₄)alkylcarbonyl group.

32. The process according to claim 23, wherein the at least one polysaccharide with at least one amine group is in a cosmetic composition and is present in the composition in an amount of between 0.01% and 20% by weight relative to the total weight of the composition.

33. The process according to claim 23, wherein composition b) comprising the at least one polysaccharide with at least one amine group has an acidic pH.

34. The process according to claim 23, wherein the at least one polysaccharide with at least one amine group has an average molecular weight MW of less than or equal to 400 kDa.

35. The process according to claim 23, wherein steps (i) and (ii) are carried out simultaneously or by applying, to the keratin fibers, a composition chosen from:
the composition (C′) having an acidic pH which comprises:
a) at least one salified monosaccharide comprising at least one amine group;
b) at least one salified polysaccharide comprising at least one amine group;
the composition (C″) having an acidic pH which comprises:
a) at least one non-salified monosaccharide comprising at least one amine group; and
b) at least one salified polysaccharide comprising at least one amine group
the composition (C′″) having an acidic pH which comprises:
a) at least one salified monosaccharide comprising at least one amine group as defined in any one of claims 1 to 5; and
b) at least one non-salified polysaccharide comprising at least one amine group; or
the composition (C″″) having an acid pH which comprises:
a) at least one non-salified monosaccharide comprising at least one amine group;
b) at least one non-salified polysaccharide comprising at least one amine group; and
c) at least one organic or mineral acid.

36. The process according to claim 23, wherein the process comprises:
(i) applying to said fibers a) at least one monosaccharide with at least one amine group; then
(ii) applying to said fibers b) at least one polysaccharide with at least one amine group; then
(iii) heat treatment above or equal to 80° C.;
wherein ingredients a) and b) are present in different cosmetic compositions.

37. The process according to claim 23, wherein the step of heat treatment or step of heating said fibers (iii) is carried out at a temperature of at least 100° C.

38. The process according to claim 23, wherein the process does not use an oxidized polysaccharide.

39. The process according to claim 23, wherein the step (ii′) of drying the keratin fibers is carried out before the heat treatment step (iii), and wherein the drying is carried out using a hairdryer, a hood, a towel, an absorbent, or by natural drying.

40. An acidic composition (C) wherein the composition comprises: either
a) at least one salified monosaccharide with at least one amine group;
b) at least one salified polysaccharide with at least one amine group; and
optionally c) at least one mineral or organic acid;
or
a) at least one non-salified monosaccharide with at least one amine group;
b) at least one salified polysaccharide with at least one amine group; and
optionally c) at least one mineral or organic acid;
and
either a) at least one salified monosaccharide with at least one amine group;
b) at least one non-salified polysaccharide with at least one amine group;
and
c) at least one mineral or organic acid, wherein the at least one mineral or organic acid is different from the following acids: aspartic acid, glutamic acid, gluconic acid, pyrrolidonecarboxylic acid, 100 OE and 500 OE ethoxylated stearic acid, linoleic acid, succinic acid and alginic acid;
or
a) at least one non-salified monosaccharide with at least one amine group;
b) at least one non-salified polysaccharides with at least one amine group as defined in any one of claims 1, 6 to 12; and
c) at least one mineral or organic acid, wherein the acid is different from the following acids: aspartic acid, glutamic acid, gluconic acid, pyrrolidonecarboxylic acid, 100 OE and 500 OE ethoxylated stearic acid, linoleic acid, succinic acid, and alginic acid.

41. The composition according to claim 40, wherein the composition does not comprise oxidized polysaccharide.

42. A multi-compartment kit comprising:
a first cosmetic composition comprising a) at least monosaccharide with at least one amine group, and optionally c) at least one organic or mineral acid;
a second cosmetic composition comprising b) at least one polysaccharide with at least one amine group, and optionally c) at least one organic or mineral acid; and
a device for heating the keratin fibers at a temperature of at least 80° C.;
wherein the first and second compositions are each packaged in a separate packaging assembly.
